(19) United States
(12) Patent Application Publication
(10) Pub. No.: US 2010/0121247 A1
(43) Pub. Date: May 13, 2010

(54) Dialysis Graft with Thromboses Prevention Arrangement

(76) Inventors: Chen Yang, Guishan Shiang (TW); Yen-Ni Hung, Taipei (TW); Mao-Tsun Wu, Taichung (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/270,002

(22) Filed: Nov. 13, 2008

(51) Int. Cl. A61M 1/00; A61F 2/82

(52) U.S. Cl. 604/8; 623/1.1

(57) ABSTRACT

A dialysis graft includes an artery connection section having one end connected to an artery; and a vein connection section having both ends connected to two sections of a vein respectively. Diameter of either end of the vein connection section is equal to that of the corresponding section of the vein. The other end of the artery connection section is integrally formed with the vein connection section and communicates therewith. An enlargement for preventing stenosis and thromboses is formed at the connection of the artery connection section and the vein connection section. The artery connection section is inclined at an acute angle with respect to the vein connection section at the connection and the enlargement is formed at the obtuse angle side of the connection.

### BACKGROUND OF THE INVENTION

1. Field of Invention

The invention relates to dialysis grafts and more particularly to a dialysis graft having an enlargement at its outlet to prevent thrombi formation and facilitate the blood flow through the dialysis graft so as to reduce the cost of thrombectomy and improve the quality of life among end-stage renal disease patients.

2. Description of Related Art

There are many Taiwanese patients have to receive dialysis graft implantation annually. According to the data provided by Taiwa dialysis Foundation in 2005, there are 19,649 males and 2,256 females have to receive hemodialysis respectively. Dialysis grafts are crucial to the success of dialysis treatment. This means that complications due to regular dialysis grafts may compromise the effectiveness of hemodialysis and even have a threat to the patient's life. The most complications (e.g., about 80%) related to the adverse effect of traditional dialysis grafts is thromboses at the ends of graft outlet. Therefore, the long-term patency of dialysis graft is crucial to the success of dialysis treatment. According to clinical experience, pulsation, flow rate, compliance between graft and native vessel, and blood pressure are the important factors that have to be observed when a dialysis is being taken. Physicians should stop dialysis immediately if any abnormalities are found.

A conventional dialysis graft 70 is shown in FIGS. 4 and 5. The dialysis graft 70 is elongated and has one end connected to an artery 80 and the other end connected to a vein 90. However, the conventional dialysis graft 70 is disadvantageous due to its inappropriate shape and structure based on clinical data. As a result, intimal hyperplasia induced stenosis are formed at a plurality of sites at the connection of the dialysis graft 70 and the vein 90 as detailed below.

The thrombi 100 may be formed because tissue in-growth at an injured endothelium of a blood vessel during an operation and blood platelets coagulation. Moreover, the thrombi 100 may be formed because a turbulent flow occurs at the connection of the dialysis graft 70 and the vein 90 due to vibration. In addition, the thrombi 100 may be formed because compliance is different at the connection of the dialysis graft 70 and the vein 90.

A typical solution for preventing stricture formation is to provide a flared portion at the connection site of the dialysis graft outlet that reduces the turbulent flow. However, stitching is necessary at the end of the dialysis graft and this may inevitably decrease the diameter of the outlet of the dialysis graft. As an end, the end of the dialysis graft becomes narrow again. Moreover, the stitching is difficult, very high in cost, and time consuming. Thus, the need for improvement still exists.

### SUMMARY OF THE INVENTION

It is therefore one object of the invention to provide a dialysis graft comprising an artery connection section having one end adapted to connect to an artery; and a vein connection section having both ends adapted to connect to two sections of a vein respectively, wherein a diameter of either end of the vein connection section is equal to that of the corresponding section of the vein; wherein the artery connection section is longer than the vein connection section; wherein the other end of the artery connection section is integrally formed with the vein connection section and communicates therewith; wherein the dialysis graft is substantially T-shaped; wherein an enlargement for preventing thromboses is formed at the connection of the artery connection section and the vein connection section; wherein the artery connection section is inclined at an acute angle with respect to the vein connection section at the connection; and wherein the enlargement is formed at the obtuse angle side of the connection.

The invention has the following advantages as compared with the prior art. The substantially T-shaped dialysis graft of the invention can prevent thrombi from being formed inside a blood vessel, thereby facilitating the blood flow through the circulatory system and reducing the cost of repeat thrombectomy. Moreover, diameter of either end of the vein connection section is equal to that of the corresponding section of the vein. That is, the connection of the vein connection section and either section of the vein has a smooth outer surface. This has the benefits of decreasing the difficulties of operation, decreasing operation time, and increasing the success probability of operation.

The above and other objects, features and advantages of the invention will become apparent from the following detailed description taken with the accompanying drawings.

### BRIEF DESCRIPTION OF THE DRAWINGS

FIG. 1 is a schematic perspective view of a preferred embodiment of dialysis graft according to the invention connecting to a vein;

FIG. 1A is a detailed view of the area in circle A in FIG. 1;

FIG. 2 is a schematic perspective view of the dialysis graft interconnecting an artery and the vein with blood flow being illustrated;

FIG. 3 is an exploded view of FIG. 2;

FIG. 4 is a schematic perspective view of a conventional dialysis graft interconnecting an artery and a vein; and

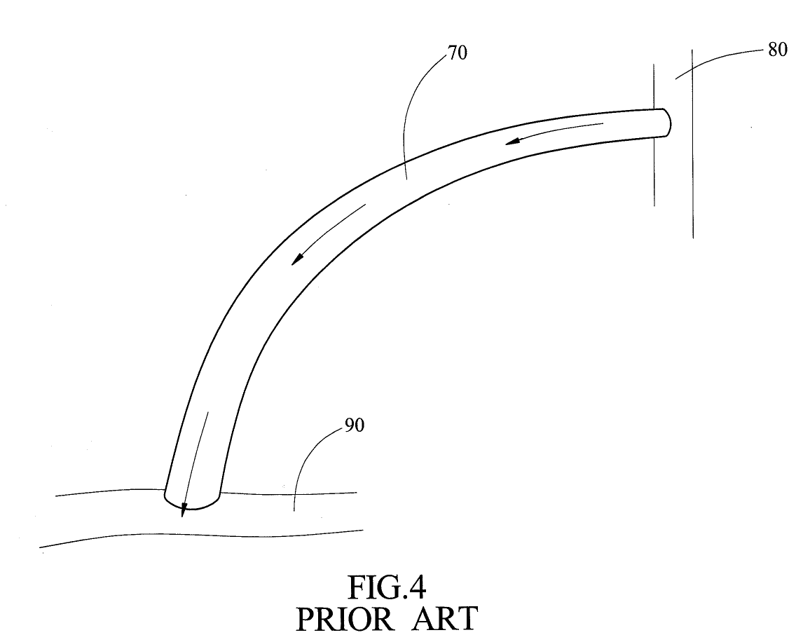

FIG. 5 is a fragmentary view in part section of FIG. 4 schematically showing the formation of thrombi at a plurality of sites at the connection of the dialysis graft and the vein.

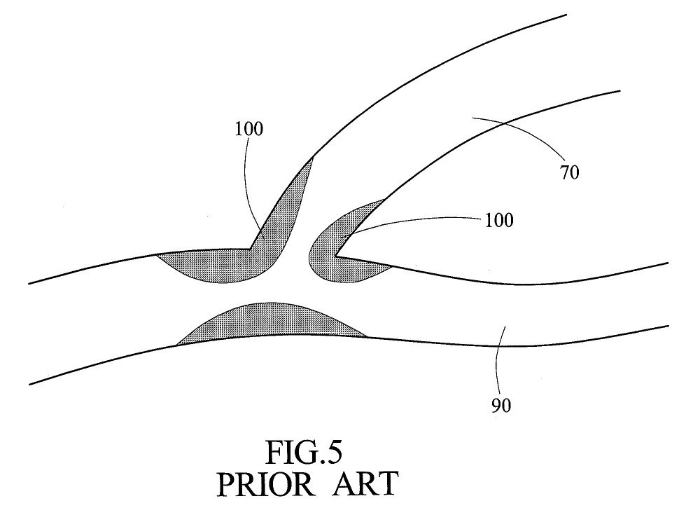

### DETAILED DESCRIPTION OF THE INVENTION

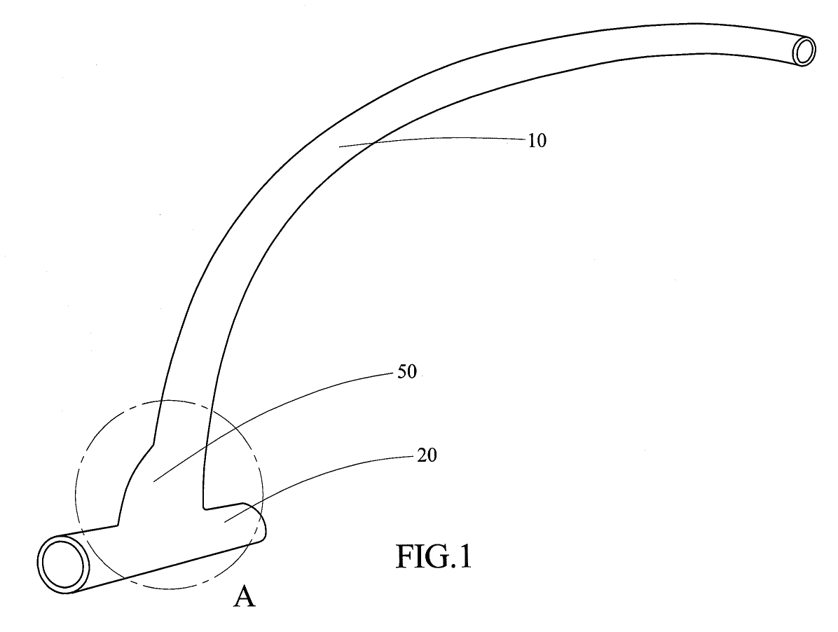

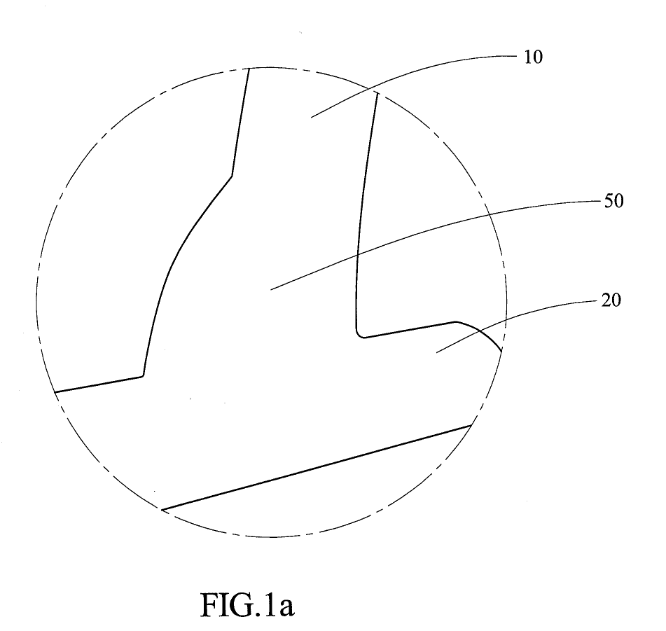

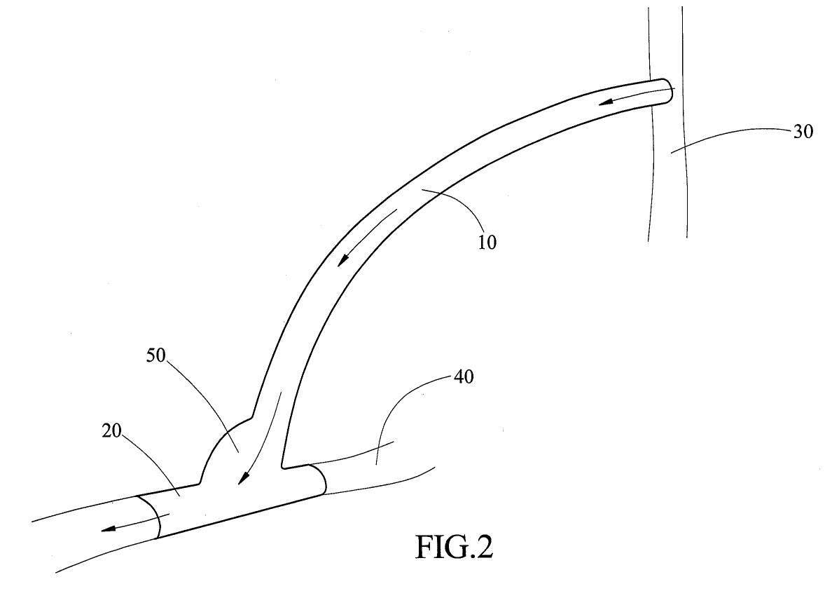

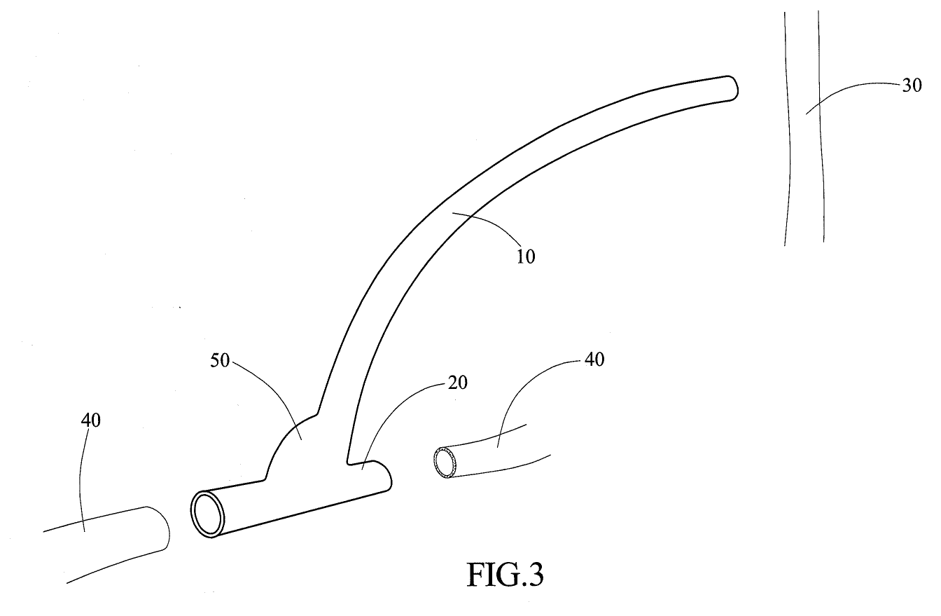

Referring to FIGS. 1 to 3, a dialysis graft in accordance with a preferred embodiment of the invention is shown. The dialysis graft comprises an artery connection section 10 and a vein connection section 20. Each of above components will now be described in detail below.

The artery connection section 10 has one end adapted to connect to an artery 30 and the vein connection section 20 has both ends adapted to connect to two sections of a vein 40 respectively. Diameter of either end of the vein connection section 20 is equal to that of the corresponding section of the vein 40. That is, the connection of the vein connection section 20 and either section of the vein 40 has a smooth outer surface. The other end of the artery connection section 10 is formed with the vein connection section 20 and communicates therewith. An enlargement 50 is formed at the connection of the artery connection section 10 and the vein connection section 20. The enlargement 50 is capable of preventing thrombi from being formed at the connection of the artery connection section 10 and the vein connection section 20. Preferably, the artery connection section 10 is longer than the vein connection section 20.

Preferably, the artery connection section 10 and the vein connection section 20 are integral.

Preferably, the dialysis graft is substantially T-shaped.

Preferably, the artery connection section 10 is inclined at an acute angle with respect to the vein connection section 20 at the connection.

Preferably, the enlargement 50 is formed at the obtuse angle side of the connection.

The invention has the following advantages. The novel design of dialysis graft can prevent thrombi formation, facilitate the blood flow through the circulatory system and reduce the cost of repeat thrombectomy. Moreover, diameter of either end of the vein connection section 20 is equal to that of the corresponding section of the vein 40. That is, the connection of the vein connection section 20 and either section of the vein 40 has a smooth outer surface. This has the benefits of decreasing the difficulties of operation, decreasing operation time, and increasing the success probability of operation.

While the invention herein disclosed has been described by means of specific embodiments, numerous modifications and variations could be made thereto by those skilled in the art without departing from the scope and spirit of the invention set forth in the claims.

What is claimed is:

1. A dialysis graft comprising:
an artery connection section having one end adapted to connect to an artery; and
a vein connection section having both ends adapted to connect to two sections of a vein respectively,
wherein a diameter of either end of the vein connection section is equal to that of the corresponding section of the vein;
wherein the other end of the artery connection section is integrally formed with the vein connection section and communicates therewith;
wherein the artery connection section is longer than the vein connection section; and
wherein an enlargement for preventing thromboses is formed at the connection of the artery connection section and the vein connection section.

2. The dialysis graft of claim 1, wherein the dialysis graft is substantially T-shaped.

3. The dialysis graft of claim 1, wherein the artery connection section is inclined at an acute angle with respect to the vein connection section at the connection, the enlargement being formed at the obtuse angle side of the connection.
